(19) United States
(12) Patent Application Publication
(10) Pub. No.: US 2017/0014310 A1
(43) Pub. Date: Jan. 19, 2017

(54) ENTERAL FEEDING ADAPTER AND METHOD OF USE

(71) Applicant: MEDELA HOLDING AG, Baar (CH)

(72) Inventors: Dongchul D. Hyun, Brea, CA (US); Andy Giles, McHenry, IL (US)

(21) Appl. No.: 15/208,430

(22) Filed: Jul. 12, 2016

Related U.S. Application Data
(63) Continuation-in-part of application No. 29/554,319, filed on Feb. 10, 2016.
(60) Provisional application No. 62/192,421, filed on Jul. 14, 2015.
(60) Provisional application No. 62/293,540, filed on Feb. 10, 2016.

(51) Int. Cl. A61J 15/00; A61M 5/34; A61J 1/20

(52) U.S. Cl. CPC A61J 15/0026; A61J 1/2096; A61M 5/345; A61M 2205/276

(57) ABSTRACT

An enteral feeding adapter system for use with a bottle cap having a dispensing opening, and an oral syringe having a male syringe element. The system includes a cap adapter having a male adapter element configured to be insertable into the dispensing opening to place the cap adapter in fluid communication with the bottle cap. The cap adapter further includes a male ENFit connector in fluid communication with the male adapter element. The enteral feeding adapter system further includes a syringe adapter having a female ENFit connector configured to be selectively engageable with the male ENFit connector to place the syringe adapter in fluid communication with the cap adapter. The syringe adapter further includes a female adapter element in fluid communication with the female ENFit connector and configured to receive the male syringe element to place the syringe adapter element in fluid communication with the oral syringe.

### CROSS-REFERENCE TO RELATED APPLICATIONS

This application claims the benefit of U.S. Provisional Application No. 62/293,540 filed Feb. 10, 2016, and U.S. Provisional Application No. 62/192,421 filed Jul. 14, 2015, and U.S. Design Application No. 29/546,173, filed Nov. 19, 2015, the contents of each of the foregoing applications being expressly incorporated herein by reference.

### STATEMENT RE: FEDERALLY SPONSORED RESEARCH/DEVELOPMENT

Not Applicable

### BACKGROUND

1. Technical Field

The present disclosure relates generally to an enteral fluid delivery system, and more specifically to an ENFit adapter system for integrating ENFit connector fittings onto syringes, feeding tubes, and medicine bottle caps.

2. Description of the Related Art

Proper nutrition is critical at all stages of life for maintaining good health. However, infants and particularly neonates, may have poor voluntary intake or lack the ability to consume enough food on their own. Therefore, it is common for nutritional foods and any required medications to be delivered directly to the gastrointestinal tract of the patient, which is generally referred to as enteral delivery.

In some instances, the fluid may be delivered to the patient orally simply by placing a syringe within the patient's mouth and dispensing the fluid therein. However, in other instances, it may be critical to ensure delivery of the fluid deeper into the patient's gastrointestinal tract. Accordingly, conventional enteral delivery systems oftentimes utilize a feeding tube designed to be inserted into the patient either via the patient's nasal opening (e.g., the nose) or through the patient's mouth, wherein the feeding tube extends into the patient's gastrointestinal tract. The enteral fluid is typically delivered to the feeding tube via a syringe, wherein the syringe is filled or loaded with the desired enteral fluid in a pharmacy or upon the nursing floor. The syringe may be transported from the pharmacy to the patient for delivery to the patient via the feeding tube. The syringe and the feeding tube may include complimentary fittings or connectors to effectuate fluid communication therebetween.

Conventional enteral delivery feeding tubes and corresponding syringes typically include Luer or oral connections or other types of small bore medical connectors, which may include a male syringe tip that fits into a female connector on the feeding tube. Such Luer connections and fittings are common not only in enteral delivery systems, but also other delivery systems, such as those used on adult patients. Accordingly, there is a risk of misadministration of enteral feeding or medication by the wrong route. Along these lines, tubing misconnections may cause severe patient injury or death, since tubes with different functions can be easily connected via the standard Luer connectors.

Recognizing the problems associated with standard connections used in enteral feeding applications, the International Organization for Standardization (ISO) has created a new standard specifically for enteral connectors, i.e., ISO standard (80369-3). This new standard is referred to generally as “ENFit.” Although ENFit connectors are uniquely configured relative to conventional connectors, such as Luer connectors, most ENFit equipment developed to date is difficult to interface with existing medicine bottle filling caps, and thus, many pharmacies are reluctant to switch over to ENFit equipment. Furthermore, the use of ENFit equipment for delivering enteral fluid may result in the patient being underdosed or overdosed, which may be critical for neonates.

Accordingly, there is a need in the art for a user friendly and unique connection system for enteral feeding applications which reduces the risk of misadministration of enteral fluid delivery and allows for accurate delivery of enteral fluid to the patient. Various aspects of the present disclosure address this particular need, as will be discussed in more detail below.

### BRIEF SUMMARY

There is provided an ENFit adapter system specifically configured to interface a syringe with a feeding tube for neonatal enteral feeding. The ENFit configuration provides a unique fitting for syringes and feeding tubes used in enteral applications and also allows for accurate administration of enteral fluid to the patient.

According to one embodiment, there is provided an enteral feeding adapter system for use with a bottle cap having a dispensing opening, and an oral syringe having a male syringe element. The enteral feeding adapter system includes a cap adapter having a male adapter element configured to be insertable into the dispensing opening to place the cap adapter in fluid communication with the bottle cap. The cap adapter further includes a first male ENFit connector in fluid communication with the male adapter element. The enteral feeding adapter system further includes a syringe adapter having a female ENFit connector configured to be selectively engageable with the first male ENFit connector to place the syringe adapter in fluid communication with the cap adapter. The syringe adapter further includes a female adapter element in fluid communication with the female ENFit connector and configured to receive the male syringe element to place the syringe adapter element in fluid communication with the oral syringe.

The present disclosure will be best understood by reference to the following detailed description when read in conjunction with the accompanying drawings.

### BRIEF DESCRIPTION OF THE DRAWINGS

These and other features and advantages of the various embodiments disclosed herein will be better understood with respect to the following description and drawings, in which:

FIG. 1 is a side, partial sectional view of an ENFit syringe in fluid communication with a medicine bottle via a bottle cap and an ENFit cap adapter;

FIG. 2 is a side view of the ENFit cap adapter depicted in FIG. 1;

FIG. 3 is a perspective view of the ENFit cap adapter depicted in FIG. 1;

FIG. 4 is a partial side, partial sectional view of the ENFit syringe coupled to the ENFit cap adapter, with enteral fluid being drawn into the ENFit syringe;

FIG. 5 is a partial side, partial sectional view of the ENFit cap adapter removed from the ENFit syringe;

FIG. 6 is a partial side, partial sectional view of an end cap placed on the ENFit syringe;

FIG. 7 is a partial side, partial sectional view of the end cap removed from the ENFit syringe;

FIG. 8 is a partial side, partial sectional view of the ENFit syringe coupled to a feeding tube assembly adapted for enteral delivery of the enteral fluid into a patient;

FIG. 9 is a perspective view of an ENFit syringe adapter;

FIG. 10 is an exploded side view of an oral syringe, the ENFit syringe adapter, and an end cap;

FIG. 11 is a side, partial sectional view of the oral syringe in fluid communication with a medicine bottle via a bottle cap;

FIG. 12 is a partial side, partial sectional view of the ENFit syringe adapter coupled to the oral syringe and the end cap;

FIG. 13 is a partial side, partial sectional view of the end cap removed from the ENFit syringe adapter;

FIG. 14 is an exploded perspective view of a portion of the oral syringe, the ENFit syringe adapter, and a feeding tube assembly;

FIG. 15 is partial side, partial sectional view of the ENFit syringe adapter coupled to the oral syringe, and the feeding tube assembly;

FIG. 16 an exploded perspective view of an ENFit syringe adapter and an ENFit cap adapter;

FIG. 17 is an exploded side view of an oral syringe, the ENFit syringe adapter, and the ENFit cap adapter;

FIG. 18 is a side, partial sectional view of an oral syringe in fluid communication with a medicine bottle via the ENFit syringe adapter, the ENFit cap adapter, and a bottle cap;

FIG. 19 is a partial side, partial sectional view of the oral syringe coupled to the ENFit syringe adapter, and the ENFit cap adapter removed from the ENFit syringe adapter;

FIG. 20 is a partial side, partial sectional view of an end cap placed on the ENFit syringe adapter, which remains coupled to the oral syringe;

FIG. 21 is a partial side, partial sectional view of the end cap removed from the ENFit syringe adapter, which remains coupled to the oral syringe;

FIG. 22 is a partial side, partial sectional view of a feeding tube assembly coupled to the ENFit syringe adapter, which remains coupled to the oral syringe;

FIG. 23 illustrates an exploded perspective view of a portion of an oral syringe, a low dose ENFit syringe adapter including an optional tamper-proof device, and a feeding tube assembly;

FIGS. 24-26 illustrate various views of an embodiment of a low dose ENFit syringe adapter; and

FIG. 27 illustrates a tamper-proof embodiment of a low dose ENFit syringe adapter.

Common reference numerals are used throughout the drawings and the detailed description to indicate the same elements.

### DETAILED DESCRIPTION

The detailed description set forth below in connection with the appended drawings is intended as a description of certain embodiments of an enteral delivery system and is not intended to represent the only forms that may be developed or utilized. The description sets forth the various structure and/or functions in connection with the illustrated embodiments, but it is to be understood, however, that the same or equivalent structure and/or functions may be accomplished by different embodiments that are also intended to be encompassed within the scope of the present disclosure. It is further understood that the use of relational terms such as first and second, and the like are used solely to distinguish one entity from another without necessarily requiring or implying any actual such relationship or order between such entities.

Various aspects of the present invention are directed toward an enteral delivery system wherein ENFit connectors are used in connection with a syringe, a feeding tube, and a medicine bottle. The use of ENFit connectors throughout the process of administering the drug or nutrients to the patient, i.e., from the transition of contents from the medicine bottle to the syringe, and then subsequently from the syringe to the patient, which may or may not include the use of a feeding tube, mitigates misconnection with tubing connections associated with tubing serving other functionalities.

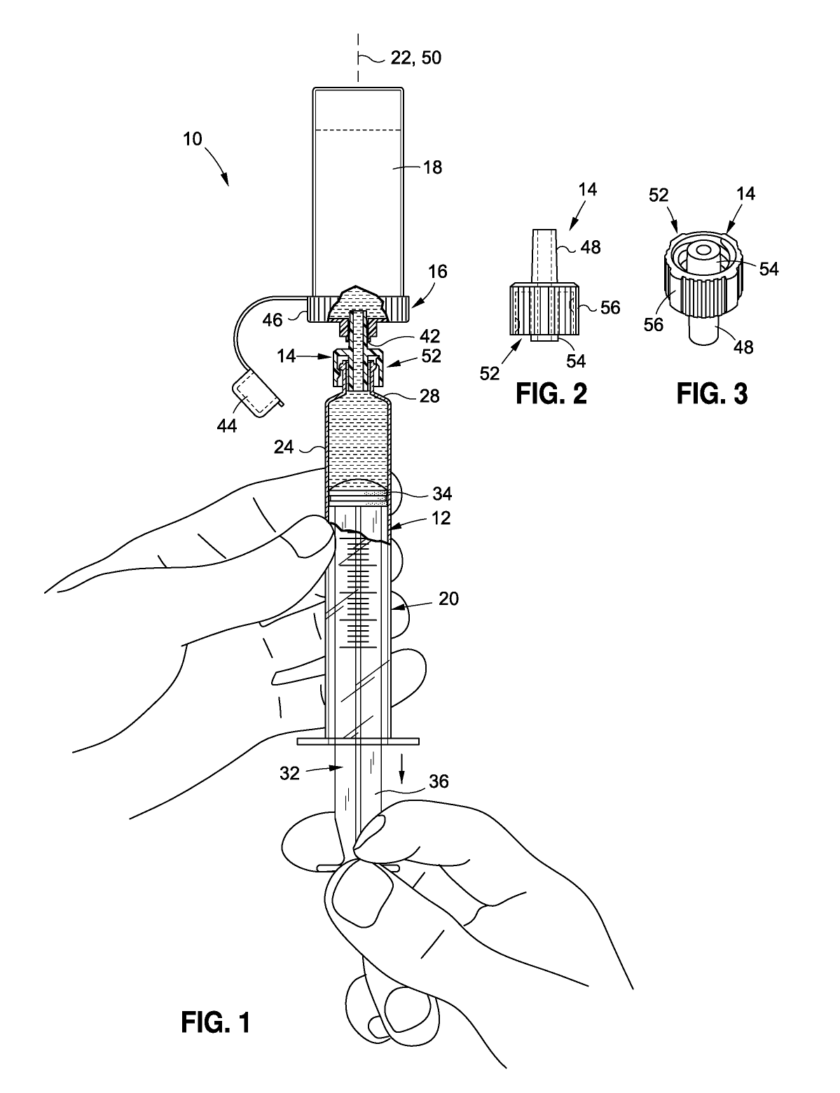

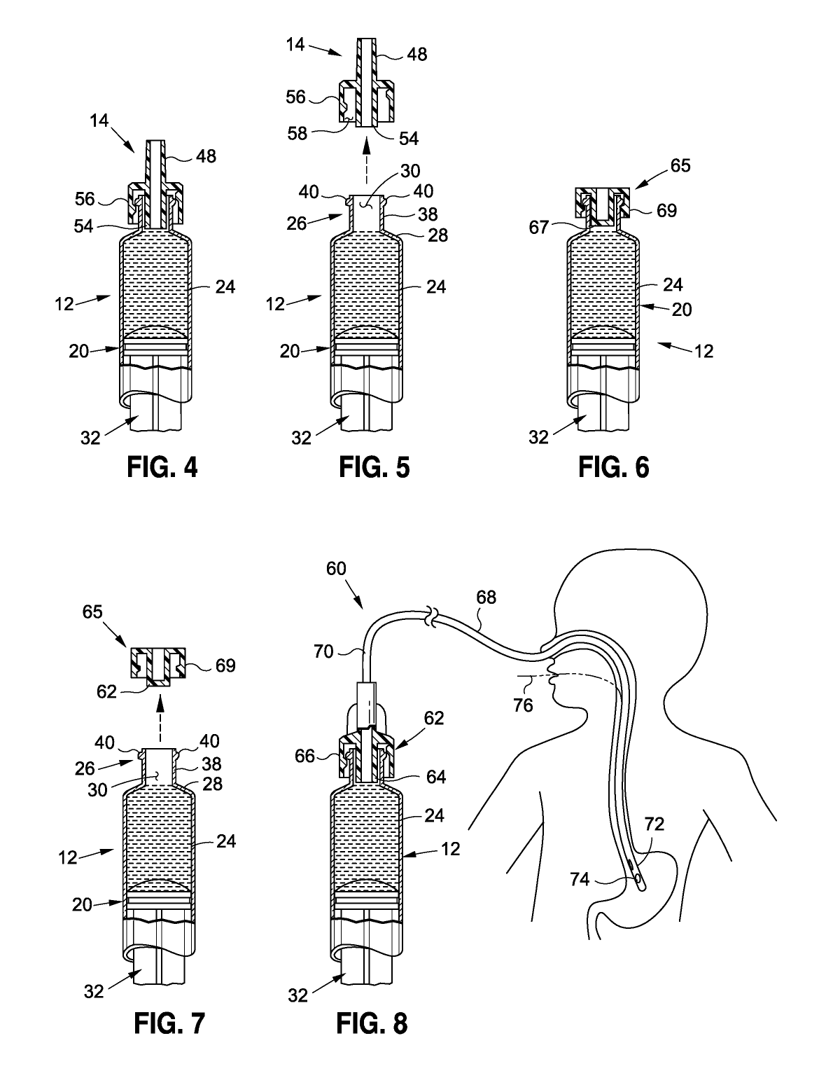

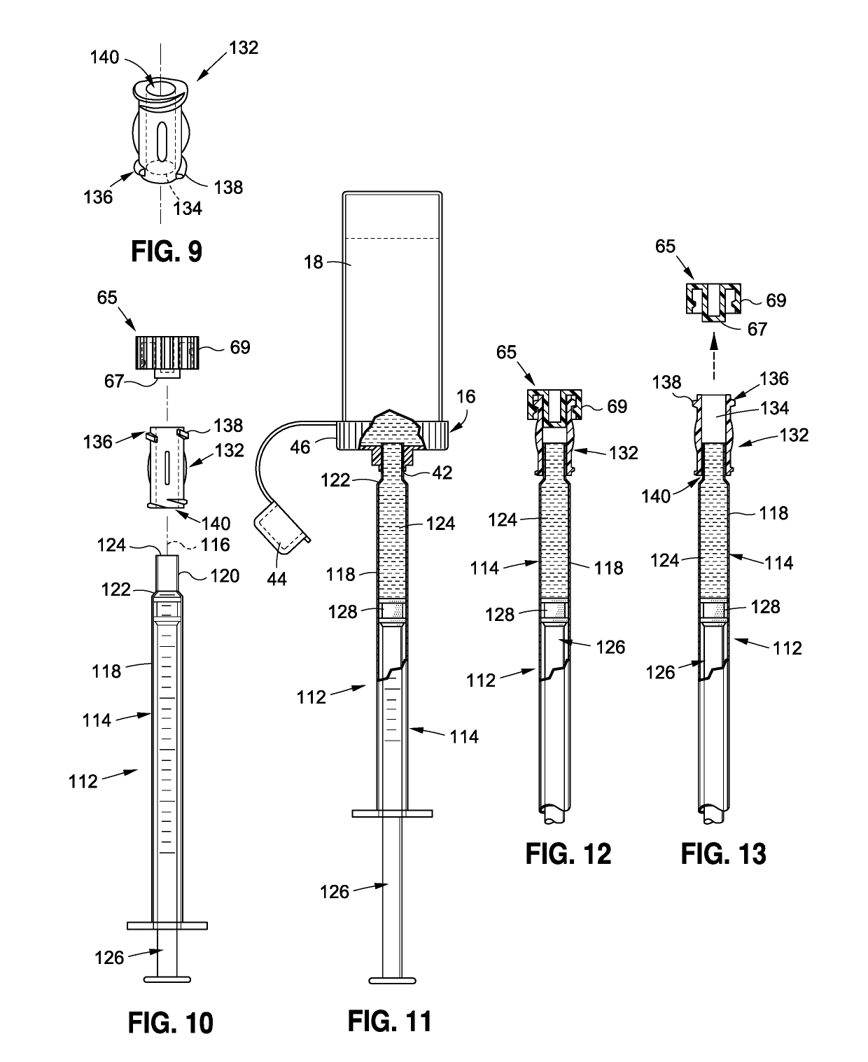

As will be described in more detail below, several embodiments of ENFit enteral adapter systems have been developed. A first embodiment of the system 10 is shown in FIGS. 1-8, and includes an ENFit syringe 12, while second and third embodiments of the system are shown in FIGS. 9-22, and include adapters configured for use with a conventional oral syringe. The use of one embodiment over the other may depend on the type of dosing that is to be conducted, e.g., dosing orally or via a feeding tube, as well as considerations in how existing delivery systems will be replaced with the embodiments described herein, as certain embodiments may be more easily implemented by pharmacists, while other embodiments may be more easily implemented by nurses or those administering the enteral fluid to the patient.

It is understood that the enteral feeding systems described herein may be used to deliver a wide range of contents to an infant, including medicine, food, or other nutritional fluids. Such contents will be collectively referred to herein as “enteral fluid” for purposes of simplicity.

A first embodiment of an enteral feeding system 10 includes an ENFit syringe 12 and an ENFit cap adapter 14 specifically adapted for use with a medicine bottle cap 16 of a medicine bottle 18, the ENFit cap adapter 14 being configured to interface the ENFit syringe 12 with the medicine bottle cap 16.

The ENFit syringe 12 includes a syringe body 20 defining a syringe axis 22 and having a cylindrical wall 24 and a female ENFit connector 26 (see FIGS. 5 and 7) both being disposed about the syringe axis 22 and separated from each other by a shoulder 28. The syringe body 20 includes a syringe opening 30 extending along the syringe axis 22, with the syringe opening 30 (see FIGS. 5 and 7) being of a first diameter at the female ENFit connector 26 and a larger second diameter at the cylindrical wall 24. The cylindrical wall 24 includes graduated markings to denote volume levels of enteral fluid drawn into the ENFit syringe 12. A plunger 32 is insertable into the syringe opening 30 at the end of the cylindrical wall 24 opposite the female ENFit connector 26 and is moveable along the syringe axis 22 within the syringe body 20 in a conventional operational manner. The plunger 32 includes a plunger head 34 having a seal that engages with an inner surface of the cylindrical wall 24 and a plunger body 36 extending from the plunger head 34 and out of the cylindrical wall 24 to enable a user to grasp the plunger body 36. The plunger 32 is moveable from a first position toward a second position, wherein the plunger head 34 is moved away from the female ENFit connector 26, to allow enteral fluid to be drawn into the syringe opening 30. The plunger 32 is moveable from the second position toward the first position, wherein the plunger head 34 is moved toward the female ENFit connector 26, to allow enteral fluid to be dispensed from the syringe 12.

The female ENFit connector 26 includes connector wall 38 disposed about the syringe axis 22, and a pair of diametrically opposed external connector locking tabs 40, which are used to secure the syringe 12 to a feeding tube or adapter cap, as will be described in more detail below. The female ENFit connector 26 used on the syringe 12 is a departure from a conventional male-type connector, which had been widely implemented on conventional oral syringes used in previous enteral feeding systems.

A standard medicine bottle cap 16 used on pharmaceutical medicine bottles 18 is shown in FIG. 1. An exemplary medicine bottle cap 16 is the Adapta-Cap™ offered by the Baxa Corporation, the structure of which is expressly incorporated herein by reference. The medicine bottle cap 16 is a threaded cap having a dispensing opening 42 to convert a standard prescription or manufacturer's bottle 16 into a filling device for oral syringes. The medicine bottle cap 16 includes a closure or cap 44 which is tethered to a main body 46 to allow the dispensing opening 42 to be closed during periods of nonuse.

The ENFit cap adapter 14 is adapted for use with both the medicine bottle cap 16 as well as the ENFit syringe 12 to enable fluid connection therebetween and to allow filling of the ENFit syringe 12 from the medicine bottle 18. The ENFit cap adapter 14 includes a male adapter element 48 configured to be insertable into the dispensing opening 42 to place the cap adapter 14 in fluid communication with the medicine bottle cap 16. According to one embodiment, the male adapter element 48 includes a straw-like member including a tapered outer surface having a minimum outer diameter less than a diameter of the dispensing opening 42 and a maximum outer diameter greater than the diameter of the dispensing opening 42.

The ENFit cap adapter 14 further defines an adapter axis 50 and includes a male ENFit connector 52 in fluid communication with the male adapter element 48, with the male ENFit connector 52 being adapted to be selectively engageable with the female ENFit connector 26 on the ENFit syringe 12 to place the male ENFit connector 52 in fluid communication with the ENFit syringe 12. The male ENFit connector 52 includes a central projection 54 configured to be at least partially advanceable into the ENFit syringe 12 when the male ENFit connector 52 is engaged with the female ENFit connector 26. An outer wall 56 is spaced radially outward from the central projection 54 to define an ENFit cavity 58 therebetween. The outer wall 56 includes internal grooves sized and configured to engage with the diametrically opposed locking tabs 40 of the female ENFit connector 26 to effectuate connection between the male and female ENFit connectors 52, 26. The outer wall 56 may further include a ribbed outer surface to facilitate gripping by a user.

The ENFit syringe 12 may be used to dispense enteral fluid into a patient via a feeding tube assembly 60 (see FIG. 8) having a specifically configured male ENFit connector 62 adapted to mate with the female ENFit connector 26 on the ENFit syringe 12. The male ENFit connector 62 of the feeding tube assembly 60 is similar to the male ENFit connector 52 on the cap adapter 14. In particular, the male ENFit connector 62 includes a central projection 64 configured to be at least partially advanceable into the ENFit syringe 12 when the male ENFit connector 62 is engaged with the female ENFit connector 26. An outer wall 66 is spaced radially outward from the central projection 64 to define an ENFit cavity therebetween. The outer wall 66 includes internal grooves sized and configured to engage with the diametrically opposed locking tabs 40 of the female ENFit connector 26 to effectuate connection between the male and female ENFit connectors 62, 26. The outer wall 66 may further include a ribbed outer surface.

A feeding tube 68 is fluidly coupled to the male ENFit connector 62 and includes a first end portion 70 coupled to the male ENFit connector 62 and an opposing second end portion 72 which may be advanced into the gastrointestinal tract of the patient. One or more openings 74 may be formed on the feeding tube 68 to allow the enteral fluid to be dispensed therefrom.

With the basic structure of the first embodiment of the enteral adapter system 10 described above, operation of the first embodiment will now be explained. The medicine bottle 18 containing the desired enteral fluid is located and a medicine bottle cap 16 is placed on the bottle 18, if a cap 16 is not already on the bottle 18. The closure 44 is opened to expose the dispensing opening 42, and an ENFit cap adapter 14 is inserted into the dispensing opening 42. In particular, the male adapter element 48 is aligned with the dispensing opening 42 and is inserted therein until the outer surface of the male adapter element 48 engages with the surface(s) of the medicine bottle cap 16 defining the dispensing opening 42. The male adapter element 48 is pressed into the dispensing opening 42 until a sufficient friction-type fit exists between the male adapter element 48 and the medicine bottle cap 16 to create a substantially fluid-tight seal therebetween and to allow for transfer of enteral fluid into the syringe 12 via the ENFit cap adapter 14.

The ENFit syringe 12 is prepared for use by ensuring the plunger 32 is in the first position. The ENFit syringe 12 is then mated with the ENFit cap adapter 14 by aligning the locking tabs 40 on the female ENFit connector 26 with the grooves on the male ENFit connector 52, and then twisting the ENFit syringe 12 relative to the ENFit cap adapter 14, which causes the female ENFit connector 26 to be advanced into the ENFit cavity 58, and the male projection 54 to be advanced into the syringe opening 30.

With the ENFit syringe 12 coupled to the ENFit cap adapter 14, the medicine bottle 18 may be inverted to place the enteral fluid adjacent the medicine bottle cap 16 and to immerse an end portion of the male adapter element 48 in the enteral fluid. The plunger 32 is then moved from the first position toward the second position, which draws enteral fluid through ENFit cap adapter 14 and into the ENFit syringe 12. The ENFit syringe 12 is then disconnected from the ENFit cap adapter 14 by twisting the ENFit syringe 12 in a second direction until the ENFit syringe 12 is released from the ENFit cap adapter 14 (see FIG. 5). An ENFit end cap 65 may be placed over the open end of the female ENFit connector 26 during transport of the ENFit syringe 12 from the pharmacy to the patient (see FIG. 6). The ENFit end cap 65 is configured similarly to an ENFit male connector, and includes a central projection 67 adapted to extend into the female ENFit connector 26 when the ENFit end cap 65 is mated thereto. The ENFit end cap 65 also includes an outer wall 69 spaced radially outward from the central projection 67 to define an ENFit cavity therebetween sized and configured to accommodate the female ENFit connector 26. The ENFit end cap 65 is coupled to the female ENFit connector 26 by twisting the ENFit end cap 65 in a first direction relative to the female ENFit connector 26. The ENFit end cap 65 may be disconnected from the female ENFit connector 26 by twisting the ENFit end cap 65 in an opposing second direction relative to the female ENFit connector 26 (see FIG. 7).

After the enteral fluid has been drawn into the ENFit syringe 12, the enteral fluid may be administered to the patient, either via a feeding tube or orally. If the enteral fluid is to be administered via a feeding tube, the ENFit syringe 12 is connected to the male ENFit connector 62 of the feeding tube assembly 60 by aligning the locking tabs 40 of the female ENFit connector 26 with the grooves formed on the outer wall 66 of the male ENFit connector 62. The female ENFit connector 26 is then twisted relative to the male ENFit connector 62, which causes the female ENFit connector 26 to be advanced into the ENFit cavity of the feeding tube assembly 60, and the male projection 64 to be advanced into the syringe opening 30. Once the ENFit syringe 12 is coupled to the male ENFit connector 62, the plunger 32 may be transitioned from the second position toward the first position, which causes the enteral fluid to evacuate from the ENFit syringe 12 and enter the feeding tube 68 via the male and female ENFit connectors 62, 26. Once the plunger 32 has been moved completely to the first position, all of the enteral fluid that is to be administered has been moved out of the ENFit syringe 12 and into the feeding tube 68. The ENFit syringe 12 may then be disconnected from the feeding tube assembly 60 by twisting the ENFit syringe 12 in an opposite second direction.

If, on the other hand, the enteral fluid is to be administered orally, the opening of the female ENFit connector 26 is placed in the patient's mouth while the plunger 32 is moved from the second position toward the first position. In this respect, the enteral fluid that is dispensed from the ENFit syringe 12 passes directly into the patient's mouth, which is represented by the dotted lines associated with reference numeral 76.

The use of the first embodiment of the ENFit adapter system 10 described above is associated with certain benefits and drawbacks. One benefit associated with the first embodiment of the ENFit adapter system 10 is that the system 10 may be used with existing medicine bottle caps 16. Another benefit is that there is only a slight practice change in the pharmacy; particularly, the use of the ENFit cap adapter 14 and the connection of the ENFit syringe 12 thereto. On the other hand, one particular drawback associated with the ENFit adapter system 10 is that the patient may be under-dosed when the enteral fluid is delivered orally. In particular, a portion of the enteral fluid remains captured in the syringe opening 30 between the end of the female ENFit connector 26 and the plunger head 34. The amount of enteral fluid remaining in the syringe is referred to as “deadspace.” Although the volume of the deadspace may be small, it may significant, particularly in view of the size of neonatal patients. Deadspace is typically not an issue when the enteral fluid is delivered through the feeding tube 68 since the male ENFit connector 62 extends into the female ENFit connector 26, thereby occupying the “deadspace,” rather than the enteral fluid occupying the deadspace. In view of the potential under-dosing when the enteral fluid is delivered orally, a determination may be required prior to filling the ENFit syringe 12 to account for potential under-dosing.

Another drawback with the ENFit syringe 12 is that the female ENFit connector 26 is not ideal for oral delivery, as the opening and size of the female ENFit connector 26 is larger than conventional oral syringes, and includes the locking tabs 40, which may be unconfortable for a neonatal patient.

Although the first embodiment of the ENFit adapter system 10 is shown with an ENFit syringe 12, those of ordinary skill in the art will recognize that the ENFit syringe 12 can be replaced with other syringes, such as an oral syringe, without departing from the spirit and scope of the present disclosure.

In view of the drawbacks associated with the first embodiment, other embodiments of ENFit adapter systems have been developed to alleviate some of those issues. In particular, various embodiments of ENFit adapter systems have been developed for use with conventional oral syringes.

Referring now to FIGS. 9-22, there is shown a second embodiment of an ENFit adapter system 110, which provides ENFit connections for a conventional oral syringe 112, and may also minimize the amount of deadspace during use of the system 110. The oral syringe 112, which includes a syringe body 114 defining a syringe axis 116 and having a cylindrical wall 118 and a male connector 120, both being disposed about the syringe axis 116 and separated from each other by a shoulder 122. The syringe body 114 includes a syringe opening 124 extending along the syringe axis 116, with the syringe opening 124 being of a first diameter at the male connector 120 and a larger second diameter at the cylindrical wall 118. The cylindrical wall 118 includes graduated markings to denote volume levels of enteral fluid drawn into the oral syringe 112. A plunger 126 is insertable into the syringe opening 124 at the end of the cylindrical wall 118 opposite the male connector 120 and is moveable along the syringe axis 116 within the syringe body 114 in a conventional operational manner. The plunger 126 includes a plunger head 128 having a seal that engages with an inner surface of the cylindrical wall 118 and a plunger body 130 extending from the seal and out of the cylindrical wall 118 to enable a user to grasp the plunger body 130. The plunger 126 is moveable from a first position toward a second position, wherein the seal is moved away from the male connector 120, to allow enteral fluid to be drawn into the syringe opening 124. The plunger 126 is moveable from the second position toward the first position, wherein the seal is moved toward the male connector, to allow enteral fluid to be dispensed from the oral syringe 112.

An ENFit syringe adapter 132 is configured for use with the oral syringe 112 to provide an ENFit-type connector for the oral syringe 112. The ENFit syringe adapter 132 includes a syringe adapter opening 134 extending therethrough from a first end portion to a second end portion to define a fluid passageway through the ENFit syringe adapter 132. The first end portion includes a female ENFit connector 136 adapted to engage with a corresponding male ENFit connector, as will be described in more detail below. The female ENFit connector 136 includes external threads or locking tabs 138 adapted to engage with internal grooves formed on a corresponding structure associated with the male ENFit connector. The second end portion of the ENFit syringe adapter 132 includes a female adapter element 140 in fluid communication with the female ENFit connector 136 via the syringe adapter opening 134, with the female adapter element 140 being configured to be fluidly connectable to the male connector 120 of the oral syringe 112. In particular, the female adapter element 140 is adapted to receive at least a portion of the male connector 120 when the oral syringe 112 is mated with the ENFit syringe adapter 132.

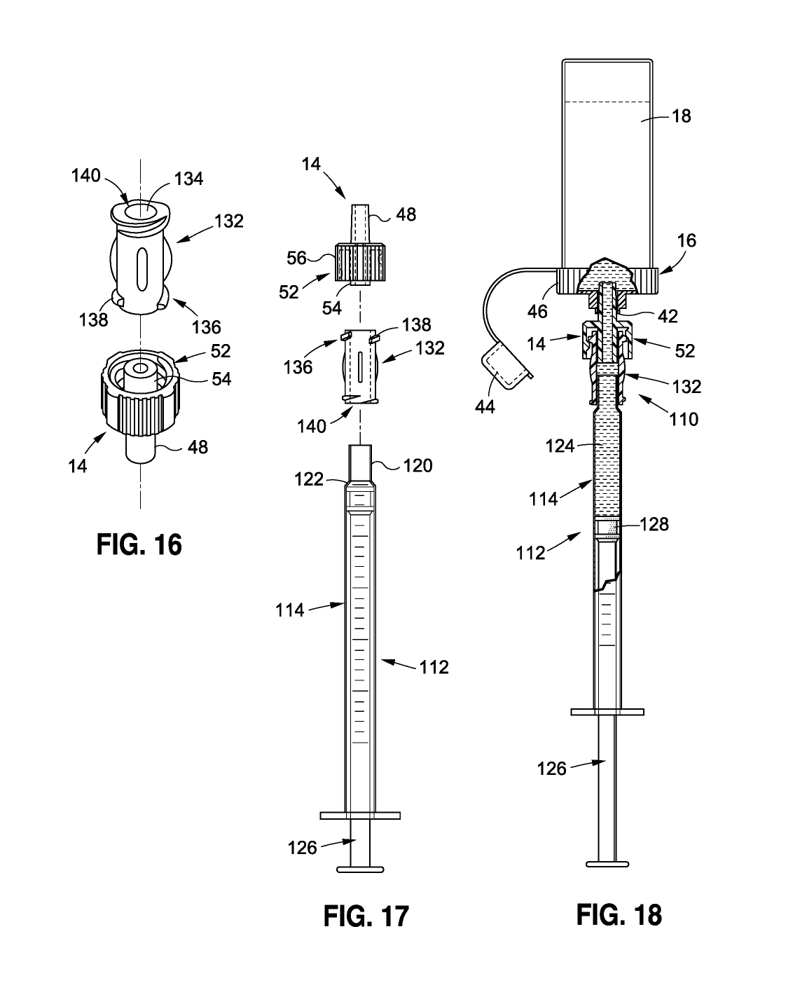

The use of the oral syringe 112 with the ENFit syringe adapter 132 for dispensing enteral fluid to a patient may be carried out using at least two different methodologies, as will be described in more detail herein. The first methodology is shown in FIGS. 11-15, while the second methodology is shown in FIGS. 16-22.

Referring now specifically to FIGS. 11-15, and according to the first methodology, the male connector 120 of the oral syringe 112 is advanced into the dispensing opening 42 of the medicine bottle cap 16 to place the oral syringe 112 in fluid communication with the medicine bottle 18. Prior to being inserted into the dispensing opening 42, the pharmacist may check to ensure the plunger 126 is in the first position, and if not, the plunger 126 may be moved to the first position. Once the male connector 120 of the oral syringe 112 is properly inserted into the dispensing opening 42 of the bottle cap 16, the oral syringe 112 and medicine bottle 18 may be inverted to place the enteral fluid adjacent the oral syringe 112, with at least a portion of the male connector 120 being immersed in the enteral fluid. The plunger 126 is then moved from the first position to the second position to draw the enteral fluid into the oral syringe 112.

Once the proper amount of enteral fluid has been drawn into the oral syringe 112, the oral syringe 112 is pulled out of the medicine bottle cap 16 (preferably after the medicine bottle 18 and oral syringe 112 have been inverted once more), and the ENFit syringe adapter 132 may be placed over the male connector 120 of the oral syringe 112, as shown in FIG. 12. Along these lines, the ENFit syringe adapter 132 is preferably configured such that the syringe adapter opening 134 is configured to at least partially receive the male connector 120, with the outer wall of the male connector 120 and the inner wall of the ENFit syringe adapter 132 creating a substantially fluid-tight seal therebetween.

An end cap 65 may be placed over the female ENFit connector 136 of the ENFit syringe adapter 132 to close off the syringe adapter opening 134, and thus, placement of the ENFit syringe adapter 132 and end cap 65 coupled thereto effectively covers or closes the dispensing end of the oral syringe 112 (e.g., the male connector 120). It is contemplated that the end cap 65 and ENFit syringe adapter 132 may be provided as a preassembled unit such that the filled syringe 112 may be capped with the dual end cap—ENFit syringe adapter assembly, rather than having to place the ENFit syringe 132 over the syringe 112, and then separately adding the end cap 65. Once the syringe 112 is filled and capped, the syringe 112 may be transported from the pharmacy to the patient for delivery of the enteral fluid.

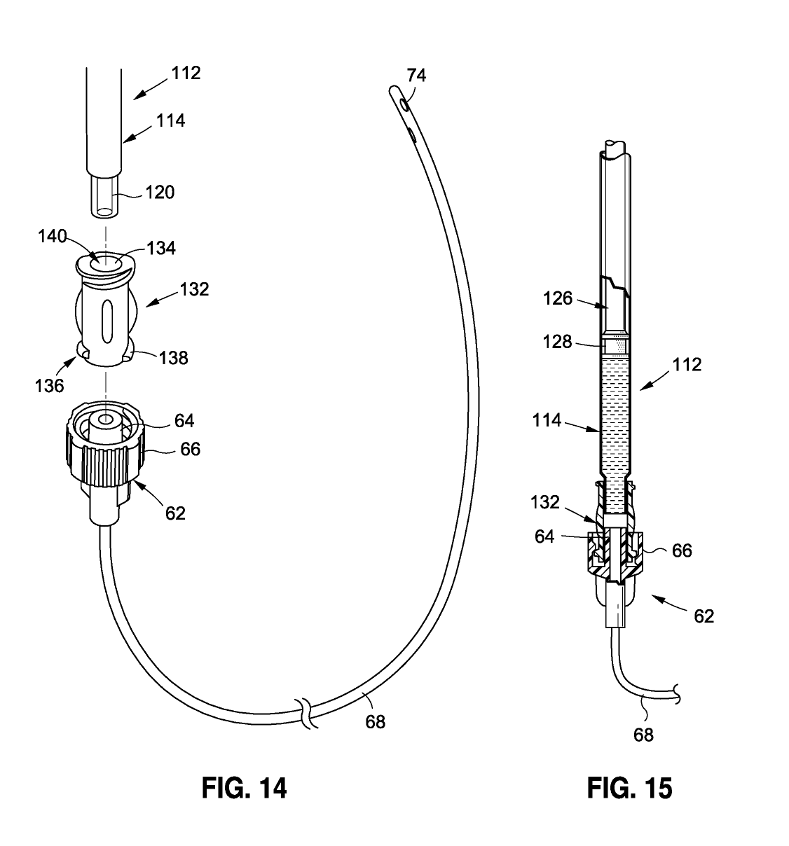

To enterally deliver the enteral fluid to the patient from the oral syringe 112 via a feeding tube 68, the end cap 65 is removed from the female ENFit connector 136 (see FIG. 13) to facilitate fluid connection of the ENFit syringe adapter 132 to the feeding tube 68. In particular, the female ENFit connector 136 is coaxially aligned with the male ENFit connector 62 coupled the feeding tube 68 by aligning the male projection 64 with the syringe adapter opening 134. The female and male ENFit connectors 136, 62 are mated by rotating the connectors 62, 136 relative to each other to allow the external locking tabs/threads 138 on the female ENFit connector 136 to engage with the internal grooves formed on the male ENFit connector 62. Such rotation also causes the male projection 64 to be advanced into the syringe adapter opening 134, such that the male and female ENFit connectors 62, 136 provide a fluid passageway between the oral syringe 112 and the feeding tube 68 (see FIGS. 14 and 15).

Once the male and female ENFit connectors 62, 136 are properly connected, the enteral fluid may be delivered to the patient by moving the plunger 126 from the second position toward the first position. Such movement pushes the enteral fluid from the oral syringe 112 and into the feeding tube 68 by way of the female ENFit connector 136 and the male ENFit connector 62. When the plunger 126 reaches the first position, all of the enteral fluid has been dispensed from the oral syringe 112. However, a small amount of enteral fluid may remain in the ENFit syringe adapter 132, and the male ENFit connector 62 of the feeding tube assembly 60, and thus, such remaining enteral fluid may not be completely delivered to the patient. Although the amount of such undelivered enteral fluid may be small, the impact to the patient may be significant, particularly for neonatal patients. Accordingly, the administrator of the enteral fluid may complete the delivery of the enteral fluid by flushing the enteral fluid into the patient. Such flushing may be accomplished by briefly detaching the oral syringe 112 from the ENFit syringe adapter 132, and drawing in air, water, or other flushing fluid into the syringe 112 by pulling the plunger 126 from the first position toward the second position when the male connector 120 is immersed within the flushing fluid. The oral syringe 112 is then reconnected with the ENFit syringe adapter 132, and the plunger 126 is moved from the second position to the first position to create fluid pressure to flush the previously undelivered enteral fluid through the feeding tube 68 for delivery to the patient.

As an alternative to delivering the medication enterally, the oral syringe 112 may also be used to deliver the enteral fluid orally. In that case, the enteral fluid may be drawn into the oral syringe 112 from the medicine bottle 18, as described above, with the syringe 112 being capped with the ENFit syringe adapter 132 and end cap 65. When the patient is ready to receive the enteral fluid, the entire assembly of the ENFit syringe adapter 132 and end cap 65 is removed from the syringe 112 and the male connector 120 is placed in the patient's mouth. The plunger 126 is then moved from the second position toward the first position to dispense the enteral fluid into the patient's mouth.

There are several benefits associated with delivery medication using the oral syringe 112 according to the first methodology described above. One benefit is that current medicine bottle caps 16 and oral syringes 112 may be used. With regard to the oral syringes 112, the smooth rounded outer contour of the male connector 120 tends to be more comfortable for a patient, whereas the locking tabs and larger diameter of the ENFit syringe may be harsh and uncomfortable for neonatal babies. Furthermore, the oral syringes 112 may be capped with the ENFit syringe adapter 132 and end cap 65 to securely close the filled syringe 112. The removal of the ENFit syringe adapter 132 and end cap 65 in the case of oral delivery is consistent with standard practices, wherein the syringes are capped in the pharmacy, and remain capped until the time of dispensing. Furthermore, the end cap 65 is removed for enteral delivery and flush, which is consistent with protocols implemented by several hospitals.

One particular drawback with the first methodology described above is that the patient may be under-dosed when the enteral fluid is administered enterally if the enteral fluid is not flushed after the initial delivery.

Referring now specifically to FIGS. 16-22, and according to the second methodology, enteral fluid is withdrawn from the medicine bottle 18 using the ENFit cap adapter 14 (described above) and the ENFit syringe adapter 132. In this respect, the ENFit cap adapter 14 is inserted into the dispensing opening 42, with the male adapter element 48 being aligned with the dispensing opening 42 and being inserted therein until the outer surface of the male adapter element 48 engages with the surfaces of the medicine bottle cap 16 defining the dispensing opening 48. The male adapter element 48 is pressed into the dispensing opening 42 until a sufficient friction-type fit exists between the male adapter element 48 and the medicine bottle cap 16 to create a substantially fluid-tight seal therebetween and to allow for transfer of enteral fluid into the oral syringe 112 via the ENFit cap adapter 14. The ENFit syringe adapter 132 is placed over the male connector 120 of the oral syringe 112, with the outer wall of the male connector 120 and the inner wall of the ENFit syringe adapter 132 creating a substantially fluid-tight seal therebetween.

The oral syringe 112 is fluidly connected to the medicine bottle 18 by mating the ENFit syringe adapter 132 with the ENFit cap adapter 14. In particular, the male projection 54 on the ENFit cap adapter 14 is aligned with the syringe adapter opening 134 and the two adapters 14, 132 are brought into contact with each other and rotated in first direction relative to each other. Such rotational motion causes the locking tabs 138 on the female ENFit connector 136 to engage with internal threads on the male ENFit connector 52, which results in axial movement of the connectors 52, 136 relative to each other, wherein the male protrusion 54 is advanced into the syringe adapter opening 134 (see FIG. 18). However, those having ordinary skill in the art will recognize that the fluid connection can be facilitated by any combination of elements or similar elements described herein.

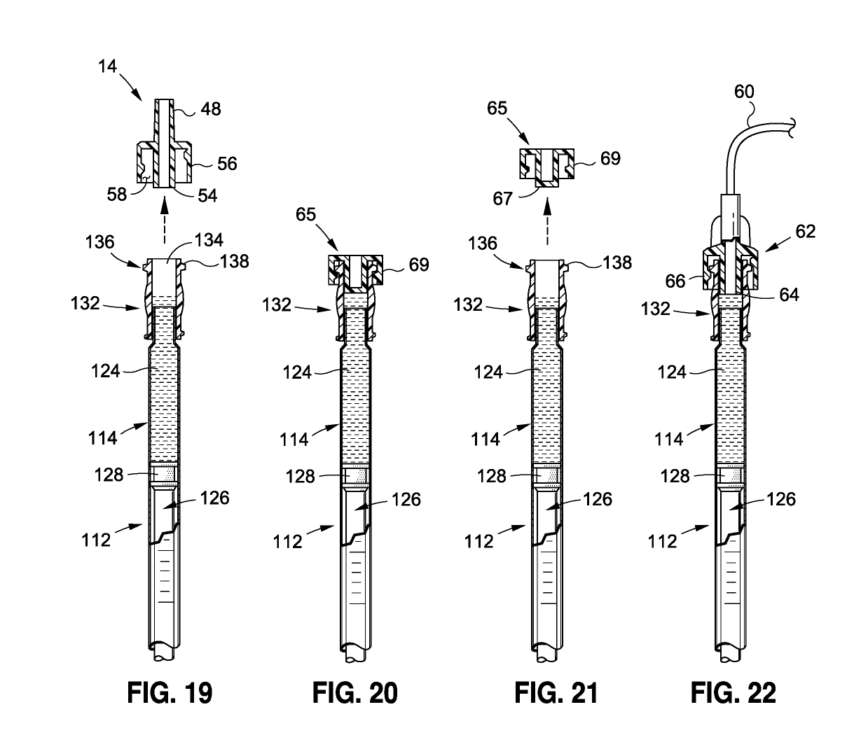

Once the male and female ENFit connectors 52, 136 are mated, the medicine bottle 18 and oral syringe 112 may be inverted and the syringe plunger 126 is pulled back from the first position to the second position to draw enteral fluid into the syringe 112. Once the proper amount of enteral fluid has been drawn into the syringe 112, the male and female ENFit connectors 52, 136 are disconnected from each other (see FIG. 19) by rotating the connectors 52, 136 relative to each other in an opposite, second direction. The end cap 65 may be temporarily placed on the ENFit syringe adapter 132 over the female ENFit connector 136 (see FIG. 20) during transport of the syringe 112 from the pharmacy to the patient, with the end cap 65 being removed from the ENFit syringe adapter 132 (see FIG. 21) prior to dispensing the enteral fluid from the syringe 112.

To enterally deliver the enteral fluid to the patient, the ENFit syringe adapter 132 is coupled to the feeding tube assembly 60 by aligning the male ENFit connector 62 of the feeding tube assembly 60 with the female ENFit connector 136 of the ENFit syringe adapter 132 and twisting the connectors 62, 136, as described in more detail above. Once the male and female ENFit connectors 62, 136 are coupled to each other, the oral syringe 112 is in fluid communication with the feeding tube 68. The plunger 126 is then moved from the second position toward the first position, which causes the enteral fluid to travel from the oral syringe 112 and into the feeding tube 68 for delivery to the patient.

When the enteral fluid is delivered enterally using the second methodology, a small amount of enteral fluid will be undelivered and remain in the ENFit syringe adapter 132, as is similar to enteral delivery using the first methodology disclosed above. However, enteral delivery using the second methodology does not require flushing, which is required using the first methodology. Flushing is not required using the second methodology because the enteral fluid is withdrawn into the syringe 112 via the ENFit syringe adapter 132. When the enteral fluid is drawn into the syringe 112, the enteral fluid will first be drawn into the syringe adapter opening 134, and then into the syringe 112. Thus, when the enteral fluid fills the syringe 112 to the proper graduation line, a small amount of additional enteral fluid will also remain in the ENFit syringe adapter 132. That small amount of additional enteral fluid compensates for the small amount of undelivered enteral fluid that remains in the ENFit syringe adapter 132 after the plunger 126 has been moved from the second position to the first position. Therefore, the full dose of enteral fluid is delivered to the patient, and subsequent flushing is not required. In this respect, the second methodology tends to be easier for nurses or other medical professionals administering the enteral fluid to the patient.

As an alternative to enteral delivery, the enteral fluid may also be delivered orally. After the syringe 112 has been filled with enteral fluid, the ENFit syringe adapter 132 is removed from the syringe 112 to expose the male connector 120 of the oral adapter 112. At that point, the oral adapter 112 may be used according to conventional practice, wherein the dispensing end of the ENFit syringe adapter 112 is placed in the patient's mouth while the plunger 126 is moved from the second position to the first position.

There are several advantages associated with the second methodology described herein. One advantage is that current medicine bottle caps 16 and oral syringes 112 may be used, and thus, medical facilities may keep their existing equipment. Furthermore, accurate doses may be delivered enterally by using the ENFit syringe adapter 132 during administration of the drug, and by removing the ENFit syringe adapter 132 for oral delivery of the drug. As such, flushing may not be required. One possible drawback associated with the second methodology is that there may be a practice change at the pharmacy.

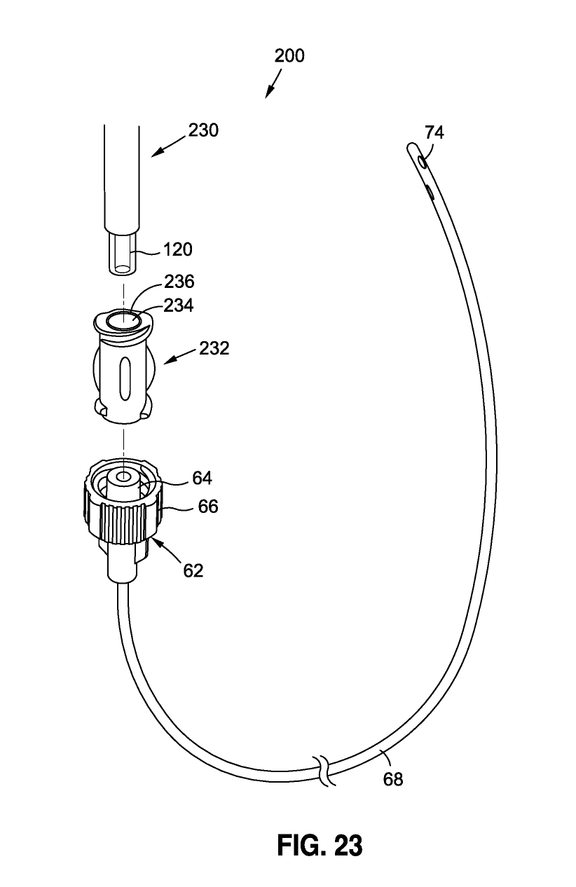

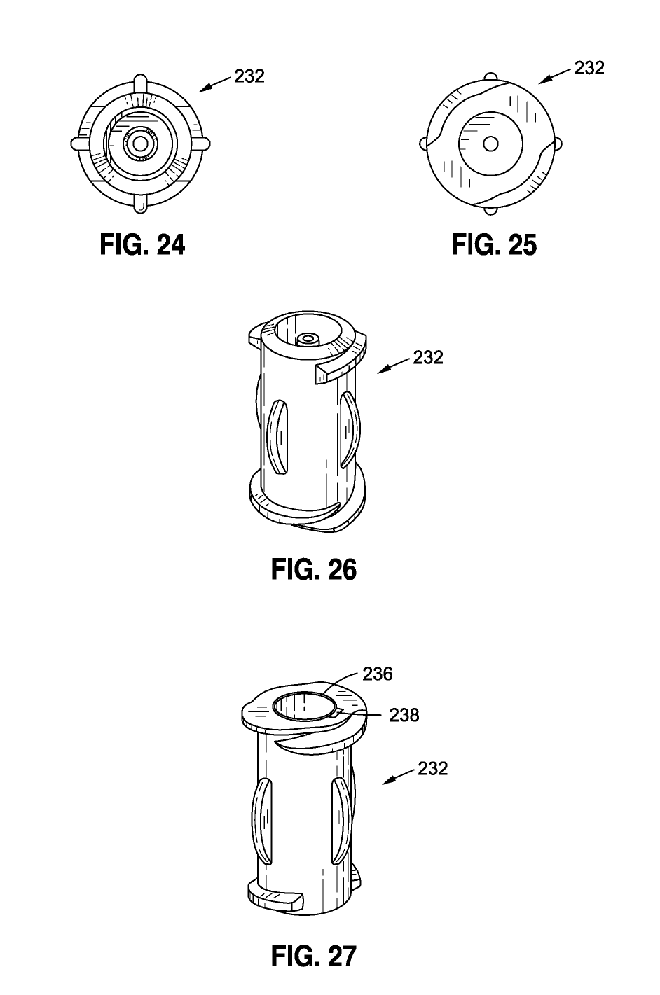

The exemplary embodiment illustrated in FIG. 23 is directed to a low dose EnFit syringe adapter solution, including an optional tamper-proof device. Additional details regarding the low dose EnFit syringe adapter are illustrated in FIGS. 24 to 26. When medication is delivered using an ENFit connection, a target dosing accuracy of +/−10% of fluid delivery volume can be introduced into the syringe adapter in a variety of ways in order to prevent under or over-delivery of the medication due to low volume dosing. In accordance with certain embodiments, the minimum fluid delivery volume can be as low as 0.2 ml, so the dosing error can be as small as 0.02 ml.

For example, the target dosing accuracy can be achieved by incorporating a structure formed in or connectable to the syringe adapter 232 to minimize the volume of fluid retained in the system. To this end, the structure, such as a suitable volume limiting member 234, can be formed within the syringe adapter 232 of the system in a number of different ways. One exemplary embodiment of a system 200 is illustrated in FIG. 23, where the volume limiting member 234 has a central flow-through aperture. The volume limiting member 234 can be integrally formed within the syringe adapter 232. The volume limiting member 234 can be configured in any suitable geometric shape, can include one or more flow-through apertures, can be integrally placed in or selectively connected to the syringe adapter 232, and can further enable the syringe adapter 232 to deliver flow therethrough at the same rate as the flow rate of the medicine through the oral syringe 230, if desired.

The system 200 can provide a delivery mechanism that enables connection within an ENFit delivery system, while ensuring the target dosing accuracy is achieved. As stated above, the syringe adapter 232 can be selectively removed from the oral syringe 230 to enable oral delivery of medicine. As illustrated in FIGS. 23 and 27, to further emphasize the correct delivery of the fluid if the syringe adapter 232 is removed by a user of the system 200, a suitable tamper-proof member, such as frangible tamper-proof member 236, for example, can be incorporated into the syringe adapter 232, such that the syringe adapter 232 is configured to provide a removable, tamper-proof connection with the oral syringe 230, and where at least one element 238 of the frangible tamper-proof member 236 can be released from the remainder of the tamper-proof member 236 when the syringe adapter 232 is removed from the oral syringe 230. As a result, a connection between the oral syringe 230 and the remaining element 238 can then indicate that the fluid can be delivered orally. The tamper-proof member and element(s) thereof can be made of any material, and can be formed in any suitable shape or size.

In the exemplary embodiment shown in FIG. 27 the tamper-proof member 236 can be configured to include at least one element 238 that separates from the syringe adapter 232 and visibly remains on the oral syringe 230 if the syringe adapter 232 is removed. Other embodiments can include imparting a visible indicator from the syringe adapter 232 onto the oral syringe 230 when the syringe adapter 232 is removed, such as by leaving a material or mark on the oral syringe 230. The material or mark then serves as an indicator that delivery of any fluid within the oral syringe is not intended for connection to a patient's IV line. The material or mark can be formulated from non-toxic food or food-like substances, if desired.

The particulars shown herein are by way of example only for purposes of illustrative discussion, and are not presented in the cause of providing what is believed to be most useful and readily understood description of the principles and conceptual aspects of the various embodiments of the present disclosure. In this regard, no attempt is made to show any more detail than is necessary for a fundamental understanding of the different features of the various embodiments, the description taken with the drawings making apparent to those skilled in the art how these may be implemented in practice.

What is claimed is:

1. An enteral feeding adapter system for use with a bottle cap having a dispensing opening, and an oral syringe having a male syringe element, the enteral feeding adapter system comprising:
a cap adapter having:
a male adapter element configured to be insertable into the dispensing opening to place the cap adapter in fluid communication with the bottle cap; and
a first male ENFit connector in fluid communication with the male adapter element; and
a syringe adapter having:
a female ENFit connector configured to be selectively engageable with the first male ENFit connector to place the syringe adapter in fluid communication with the cap adapter; and
a female adapter element in fluid communication with the female ENFit connector and configured to receive the male syringe element to place the syringe adapter element in fluid communication with the oral syringe.

2. The enteral feeding adapter system recited in claim 1, wherein the male adapter element includes a tapered outer surface having a minimum outer diameter less than a diameter of the dispensing opening and a maximum outer diameter greater than the diameter of the dispensing opening.

3. The enteral feeding adapter system recited in claim 1, wherein the cap adapter defines a cap adapter axis, the first male ENFit connector including an outer wall disposed about and spaced from the cap adapter axis.

4. The enteral feeding adapter system recited in claim 3, wherein the outer wall includes a ribbed outer surface.

5. The enteral feeding adapter system recited in claim 3, wherein the first male ENFit connector further includes a central projection spaced from the outer wall to define an ENFit cavity therebetween, the central projection being at least partially advanceable into the syringe adapter when the female ENFit connector is engaged with the first male ENFit connector.

6. The enteral feeding adapter system recited in claim 5, wherein the syringe adapter includes an opening extending therethrough defining a fluid passageway, the opening being adapted to at least partially receive the central projection when the female ENFit connector is engaged with the first male ENFit connector.

7. The enteral feeding adapter system recited in claim 3, wherein the female ENFit connector includes external threads adapted to engage with internal grooves formed on an inner surface of the outer wall of the first male ENFit connector.

8. The enteral feeding adapter system recited in claim 1, further comprising a feeding tube assembly including a feeding tube and a second male ENFit connector fluidly coupled to the feeding tube, the second male ENFit connector being adapted to be selectively engageable with the female ENFit connector to place the syringe adapter in fluid communication with the feeding tube assembly.

9. An enteral adapter for use with a bottle cap having a dispensing opening, and an ENFit syringe having a female ENFit connector, the enteral adapter comprising:
a male adapter element configured to be insertable into the dispensing opening to place the cap adapter in fluid communication with the bottle cap; and
a male ENFit connector in fluid communication with the male adapter element, the male ENFit connector being adapted to be selectively engageable with the female ENFit connector to place the male ENFit connector in fluid communication with the ENFit syringe.

10. The enteral adapter recited in claim 9, wherein the male adapter element includes a tapered outer surface having a minimum outer diameter less than a diameter of the dispensing opening and a maximum outer diameter greater than the diameter of the dispensing opening.

11. The enteral adapter recited in claim 9, wherein the male ENFit connector includes an outer wall disposed about and spaced from an axis.

12. The enteral adapter recited in claim 11, wherein the outer wall includes a ribbed outer surface.

13. The enteral adapter recited in claim 11, wherein the male ENFit connector further includes a central projection spaced from the outer wall to define an ENFit cavity therebetween, the central projection being at least partially advanceable into the ENFit syringe when the male ENFit connector is engaged with the female ENFit connector.

14. A syringe adapter comprising:
a volume limiting reservoir, the volume limiting reservoir having an outlet disposed within the syringe adapter.

15. The syringe adapter according to claim 14, further rcomprising at least one tamper-proof element.
